(19) United States
(12) Patent Application Publication
(10) Pub. No.: US 2012/0040887 A1
(43) Pub. Date: Feb. 16, 2012

(54) COMPOSITION FOR THE TREATMENT OF OSTEOMYELITIS, METHOD FOR PREPARING THE SAME, AND METHOD FOR THE TREATMENT OF OSTEOMYELITIS

(75) Inventors: Chin-Fu Chen, Sinjhuang City (TW); Hsin-Hsin Shen, Hsinchu County (TW); Yu-Min Lee, Xinying City (TW); I-Ming Chu, Hsinchu City (TW); Kuo-Ti Peng, Puzi City (TW)

(73) Assignee: INDUSTRIAL TECHNOLOGY RESEARCH INSTITUTE, Hsinchu County (TW)

(21) Appl. No.: 12/899,823

(22) Filed: Oct. 7, 2010

(30) Foreign Application Priority Data
Aug. 16, 2010 (TW) 099127259

(51) Int. Cl. A61K 38/14; A61K 31/7034; A61P 31/04; A61K 31/7036

(52) U.S. Cl. 514/2.4; 514/40; 514/39; 514/35

(57) ABSTRACT

The disclosure provides a composition for the treatment of osteomyelitis and a method for preparing the same. The composition includes: 100 parts by weight of water; 0.06-0.1 parts by weight of an antibiotic; and 5-40 parts by weight of a biodegradable thermosensitive polymer, wherein the biodegradable thermosensitive polymer has a structure as following:

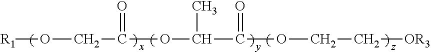

wherein, R₁ is hydrogen, or —C(═O)—R₂; R₂ is C₇₋₃₀ alkyl substituted or unsubstituted with functional groups; R₃ is hydrogen, or C₁₋₆ alkyl; and x, y or z individually are integers greater than 0.

### CROSS REFERENCE TO RELATED APPLICATIONS

This application is based upon and claims the benefit of priority from the prior Taiwan Patent Application No. 099127259, filed on Aug. 16, 2010, the entire contents of which are incorporated herein by reference.

### TECHNICAL FIELD

The disclosure relates to a biodegradable thermosensitive polymer composition, and in particular relates to a biodegradable thermosensitive polymer composition for the treatment of osteomyelitis and a method for the treatment of osteomyelitis.

### BACKGROUND

The occurrence of osteomyelitis usually results from the polyinfection of various bacteria, but the staphylococcus aureus is a major pathogen for osteomyelitis. The principles for treating the osteomyelitis are focus clearance, antibacterial action, and promotion of recovery protein hyperplasia. Currently, the treatment of the osteomyelitis includes applying a bone cement (polymethylmethacylate, PMMA)) composition with antibiotics to the affected region. Patients using the bone cement are not apt to develop allergies thereto due to the biologically inert properties thereof. Specifically, the bone cement is non-biodegradable and may become a nidus for infection. Therefore, a second operation would be applied to the patients to remove the non-biodegradable material, resulting in an extension of the course of the treatment.

### SUMMARY

The disclosure provides a composition for the treatment of osteomyelitis and a method for preparing the same. The composition includes: 100 parts by weight of water; 0.06-0.1 parts by weight of an antibiotic; and 5-40 parts by weight of a biodegradable thermosensitive polymer, wherein the biodegradable thermosensitive polymer has a structure as following:

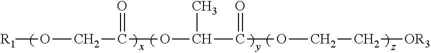

wherein, R₁ is hydrogen, or —C(═O)—R₂; R₂ is C₇₋₃₀ alkyl substituted or unsubstituted with functional groups; R₃ is hydrogen, or C₁₋₆ alkyl; and x, y or z individually are integers greater than 0.

The disclosure also provides a method for preparing the aforementioned composition for the treatment of osteomyelitis, including mixing the above components under 5-10° C., and obtaining a liquid (injectable) drug delivery system. The liquid drug delivery systems are transferred to a sol-gel drug delivery system due to environmental temperatures. The sol-gel drug delivery system is capable of near-linear controlled release of antibiotics for an extended period of time for treatments.

The disclosure also provides a method for the treatment of osteomyelitis. The method includes intramuscularly administering the aforementioned composition to a subject suffering from osteomyelitis under conditions such that said osteomyelitis is reduced.

A detailed description is given in the following embodiments with reference to the accompanying drawings.

### BRIEF DESCRIPTION OF THE DRAWINGS

The disclosure can be more fully understood by reading the subsequent detailed description and examples with references made to the accompanying drawings, wherein:

FIG. 1 is a ¹H NMR spectra of Product III (mPEGe-PLGA).

FIG. 2 is the phase diagrams of Products I-III.

FIG. 3 is a graph plotting temperature against viscosity of Products I-III.

FIG. 4A is a graph plotting residual weight against time of Product III.

FIG. 4B is a graph plotting residual molecular weight against time of Product III.

FIG. 5 is the release profiles of a composition with Product III and teicoplanin.

FIG. 6 is the histological grading scale of osteomyelitic tissues after 4 weeks and 8 weeks, respectively, under various treatments.

FIG. 7A is the expression of the COL1A1 protein of osteomyelitic tissues after 4 weeks under various treatments.

FIG. 7B is the expression of the COL1A1 protein of osteomyelitic tissues after 8 weeks under various treatments.

### DETAILED DESCRIPTION

The disclosure provides a composition for the treatment of osteomyelitis and a method for preparing the same. The composition includes: 100 parts by weight of water; 0.06-0.1 parts by weight of an antibiotic; and 5-40 parts by weight of a biodegradable thermosensitive polymer, wherein the biodegradable thermosensitive polymer has a structure as following:

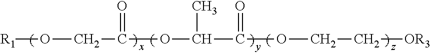

wherein, R₁ is hydrogen, or —C(═O)—R₂; R₂ is C₇₋₃₀ alkyl substituted or unsubstituted with functional groups; R₃ is hydrogen, or C₁₋₆ alkyl; and x, y or z individually are integers greater than 0.

Regarding the composition, the composition is not apt to be water-soluable when the dose of the biodegradable thermosensitive polymer is over 40 parts by weight; further, the composition is not apt to be transferred to a sol-gel state under human body temperature when the dose of the biodegradable thermosensitive polymer is below 5 parts by weight.

The repeat unit mPEG can have a molecular weight of 550 g/mole. Further, the repeat unit PLGA can have a molecular weight of 990-1540 g/mole. Moreover, the ratio between x+y and z is from 1.8 to 2.8.

In an embodiment of the disclosure, the antibiotic can include teicoplanin, vancomycin, telavancin, neomycin, or tobromycin.

The composition exhibited a solution below 10° C. and above 50° C., and the composition exhibited a sol-gel between 10-50. In practical applications, first the composition can be prepared by mixing water, antibiotic, and biodegradable thermosensitive polymer under 5-10, exhibiting an injectable solution. Next, the composition can be injected into the affected region of osteomyelitis, exhibiting a sol-gel due to the human body temperature. The drug release rate can be controlled by the in vivo hydrolysis rate of the gel. Therefore, the composition does not need to be removed by additional operations.

The following examples are intended to illustrate the disclosure more fully without limiting the scope of the disclosure, since numerous modifications and variations will be apparent to those skilled in this art.

Preparation of mPEG-PLGA

A series of the monomethoxypoly(ethylene glycol)-co-poly(lactic-co-glycolicacid) (mPEG-PLGA) diblock copolymers were synthesized by the ring-opening polymerization of monomers and mPEG in the presence of stannous 2-ethylhexanoate.

First, different amounts of mPEG (37.76 g for Product I, 30.54 g for Product II and 24.02 g for Product III) (sold by Polyscience) were mixed, respectively, with lactide (50 g, sold by PURAC biomaterial) and glycolide (11.39 g, sold by PURAC biomaterial) in a dry four-neck reactor with mechanical stirrer. The reactor temperature was controlled by an electric heater with a feedback sensor at 160° C. The catalyst, stannous 2-ethylhexanoate (38 ml for Product I, 37 ml for Product II, and 34 ml for Product III), was added in the reactor to process polymerization at 160° C. for 8 hrs.

The synthesis pathway was as follows:

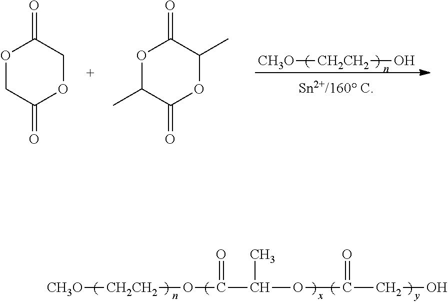

The resulting copolymer was further purified by dialysis (MWCO 1000) for 3 days at 4° C. and freeze dried by lyophilization for 5 days, obtaining Products I-III.

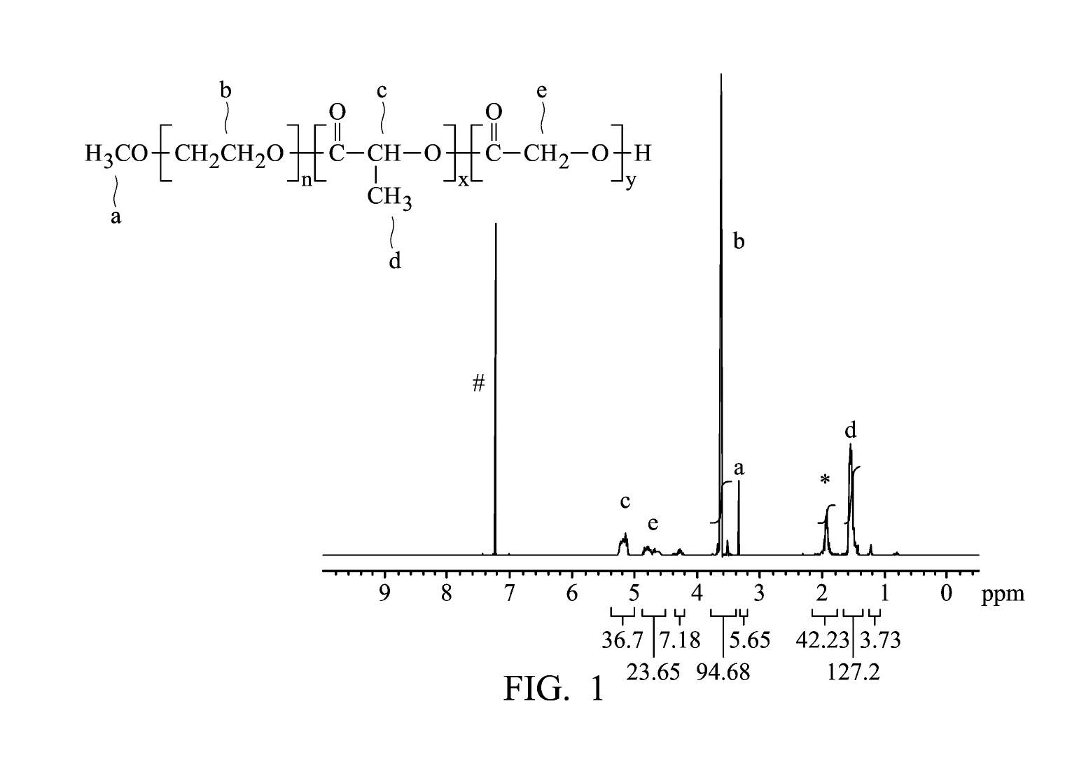

The molecular configuration of Product III was determined with an NMR spectrometer (Varian 500), as shown in FIG. 1.

The molecular weights of Products I-III were determined using a GPC (Spectra system (AS1000) P680HPLC pump and RI-150 refractive index detector coupled to a series of Plgel 5 mm column). Tetrahydrofuran (THF) served as a solvent with a flow rate of 1 ml/min. The molecular weights of Products I-III were determined relative to polyethylene glycol standards. The results are shown in Table 1.

TABLE 1

| | Theoretical | | | |
|---|---|---|---|---|
| | composition | | | |
| | of mPEG/PLGA | Mn | Mw | MW |
| Product I | 550-1030 | 1278 | 1724 | 1.35 |
| Product II | 550-1105 | 1324 | 1875 | 1.42 |
| Product III | 550-1405 | 1492 | 2037 | 1.37 |

Critical Micelle Concentration (CMC) Determination

The CMC (Critical micelle concentration) values were determined by the dye solubilization method. The hydrophobic dye 1,6-diphenyl-1,3,5-hexatriene (DPH) was dissolved in methanol with a concentration of 0.4 mM. About 200 ml of the solution was mixed with 2.0 ml of copolymer aqueous solution with concentrations ranging from 0.0001 to 1 wt % and equilibrated overnight at 4° C. A UV-VIS spectrophotometer was used to measure the absorbance at 356 nm. The CMC value was determined by the plot of the absorbance versus logarithmic concentration, as shown in Table 2.

The particle sizes of polymeric micelles were measured using dynamic light scattering (DLS). The DLS measurement was carried out on a Malvern Zeta 1000HS spectrophotometer equipped with a HeeNe laser at 633 nm at 25° C. and a fixed scattering angle of 90°. A nano-micelle solution prepared by a dialysis method was used for a particle size measurement (concentration: 1.0 wt %). The solution was first filtered through a 0.22 mm filter membrane before measurement. The results are shown in Table 2.

TABLE 2

| | CMC (mg/mL) | Particle size (nm) | Poly index |
|---|---|---|---|
| Product I | 6.7 * 10−2 | 50.8 | 0.35 |
| Product II | 5.3 * 10−2 | 62.8 | 0.35 |
| Product III | 3.2 * 10−2 | 100.1 | 0.50 |

Determination of Solegel Phase Transition

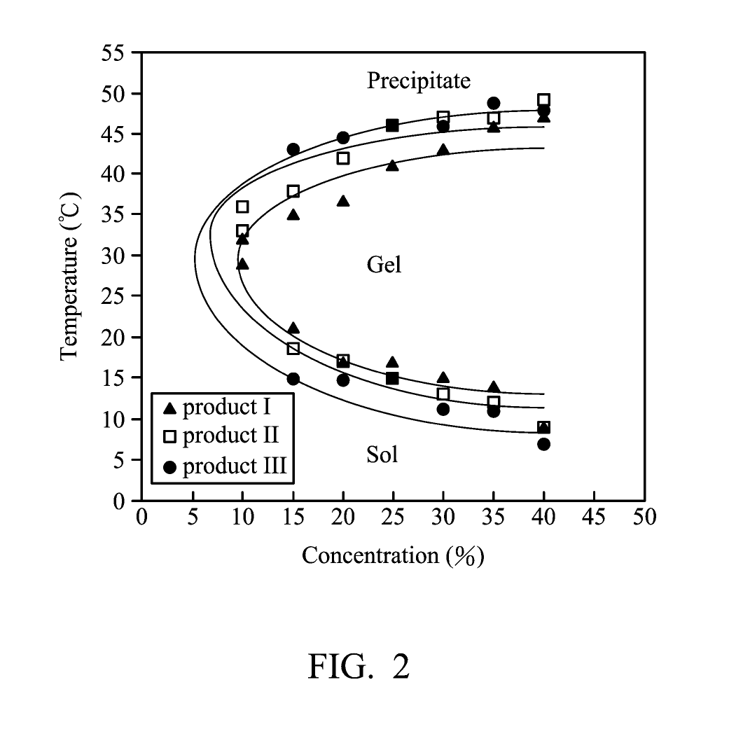

Solutions of Products I-III in deionized water with a range of concentrations (10-40 wt %) were prepared, respectively, and stored in vials at 4° C. After 24 hrs, the vials containing the polymer solutions were immersed in a water bath at 5° C., and the phase transitions of the polymer solutions were investigated by raising the bath temperature from 5° C. to 55° C. in increments of 2.5° C., holding the sample for 10 min. at each temperature. After the 10 min. equilibrium time at each temperature, the vials were tilted to determine if the diblock copolymer solution flowed. The phase transition temperature was taken as the first temperature at which the solution did not flow when tilted. The phase diagrams of Products I-III are shown in FIG. 2.

Viscosity of Sol-Gel Transition

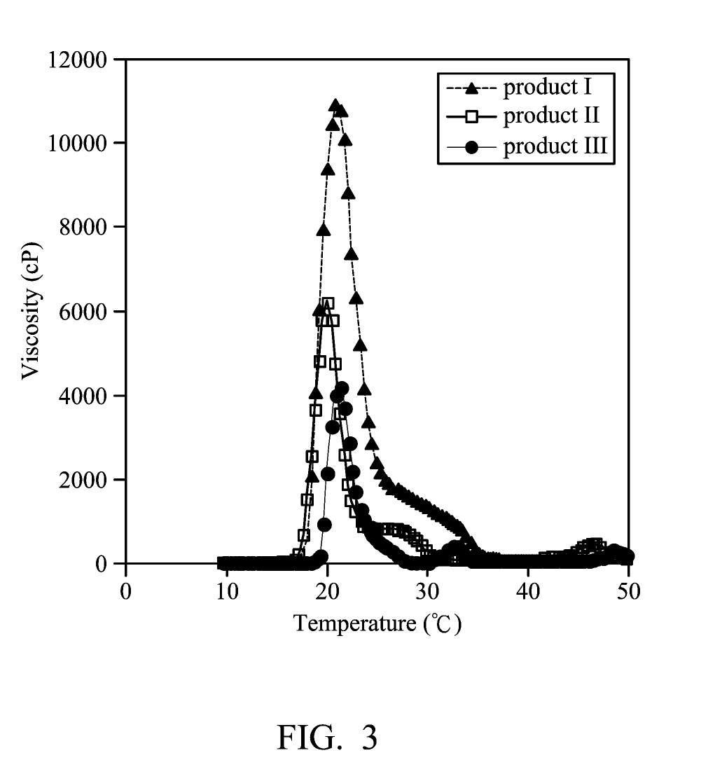

Viscosity of the Products I-III aqueous solution with various concentrations was measured by the Rheometer (Haake Rheostress RS600) with a temperature controller (TC501) at 5-50° C. The 0.5 ml polymeric solutions were stored in a corn-plate instrument with the temperature controller and subjected to the viscosity analysis, and the results are shown in FIG. 3. As shown in FIGS. 2 and 3, the mPEG-PLGA copolymer exhibited a solution state at 5-10° C., and exhibited a sol-gel state at 37° C. (human body temperature).

In Vitro Degradation of the mPEG-PLGA Copolymers

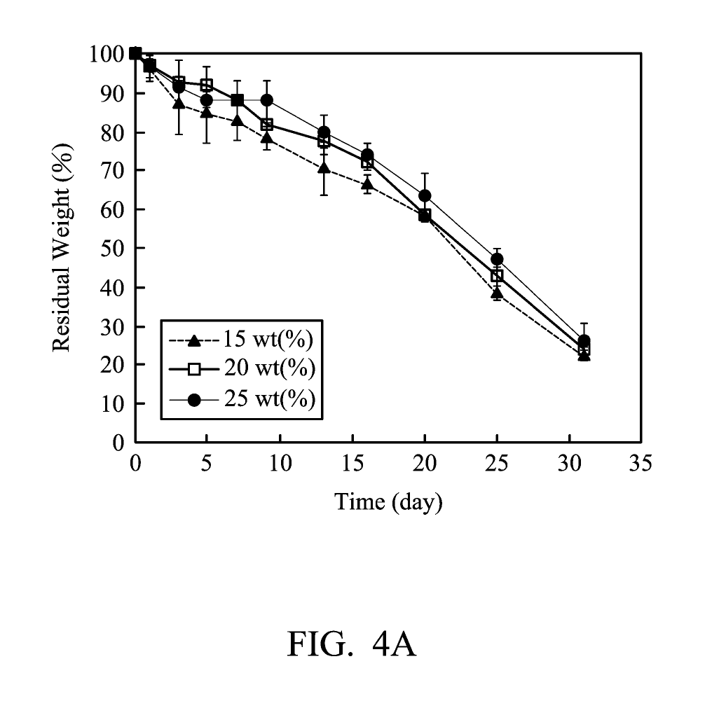

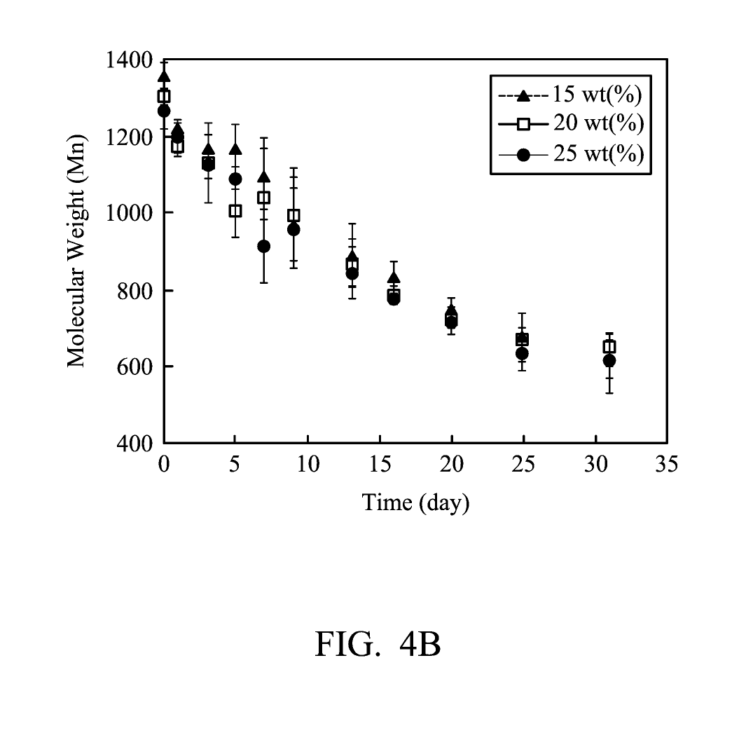

The degradation behavior of Products I-III was evaluated by mass loss and molecular weight (Mn) reduction was evaluated with time upon their in vitro incubation in phosphate buffered saline (PBS). Samples (0.5 ml) were incubated in 4 ml of PBS at 37° C. under mild agitation in a water bath. The solid residues were removed from the incubation medium at scheduled time intervals and lyophilized. The samples were weighted and the weight loss was calculated. Then, the solid residues were dissolved in THF and subjected to gel permeation chromatography (GPC) analysis to determine the average molecular weight of the samples. FIG. 4A shows a graph plotting residual weight against time of Product III, and FIG. 4B shows a graph plotting residual molecular weight of solid residues against time of Product III.

Teicoplanin Release Experiment In Vitro

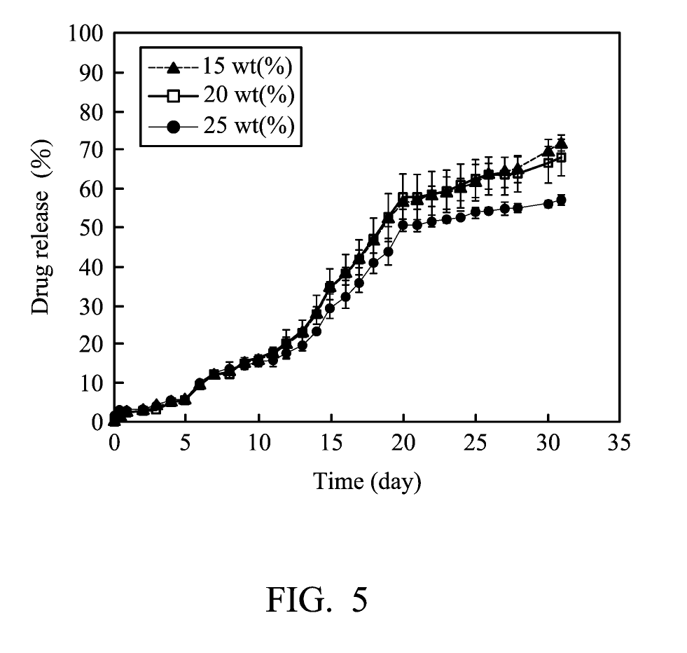

First, a composition including the mPEG-PLGA copolymer (Product III) and Teicoplanin was prepared. Teicoplanin was added to 15 wt %-, 20 wt %- and 25 wt %-copolymer aqueous solution and homogenized with Vortex mixer for 1 min. at room temperature or below. The final concentration of teicoplanin in copolymer solution was 840 μg/ml. One milliliter of the teicoplanin-containing copolymer was subsequently loaded into the bottom of 10 ml release cell and kept at 37° C. for 5 min. to form the hydrogel. For analyzing the teicoplanin release, 9 ml of PBS was added into the release cell as release medium and maintained at 37° C. in a thermostatic bath with shaking for 50 rpm. At scheduled time points, the PBS media containing teicoplanin was collected to analyze the amount of teicoplanin by HPLC. FIG. 5 shows the release profiles of a composition with Product III and Teicoplanin.

Referring to FIGS. 4A and 5, the Teicoplanin release amount increases proportionately with the degradation degree of the mPEG-PLGA copolymer. Therefore, the encapsulated Teicoplanin is gradually released by the composition during the degradation of the mPEG-PLGA copolymer. Since the teicoplanin is released in a near-linear manner, the composition of the disclosure exhibited improved drug release control, avoiding undue release of drug.

In Vivo Study

Thirty-six New Zealand white rabbits, weighing 2.5-3 kg, were selected for this study. All animal experimental protocols were approved by the Institutional Animal Care and Use Committee of the Chang Gung Memorial Hospital and carried out according to the guideline of the National Institutes of Health (Bethesda, Md.). The right femur of each rabbit was inoculated with 2×10⁶ staphylococcus aureus. The staphylococcus aureus used in this study was isolated from an osteomyelitis patient, and deposited at the American Type Culture Collection as strain ATCC 49230. Briefly, the staphylococcus aureus cells were prepared from overnight cultures grown in a tryptic soy broth at 37° C. with aeration, and then harvested by centrifugation and re-suspended to a final concentration of 2×10⁸ colony-forming units (CFU)/mL. All animals fasted for one day before bacterial inoculation. Anesthesia was induced with ketamine (25 mg/kg) and xylazine (10 mg/kg) by intravenous injection. An incision was made on a lateral surface and extended down to a distal femur. A MicroHall oscillating saw (Linvatek, Largo, Fla.) was used to create a 0.8 cm diameter bony defect from the distal shaft of the femur. Ten microliters of staphylococcus aureus (2×10⁸ CFU/ml) was injected directly into the defect of the femur.

Four weeks later, each rabbit was sent to the operating room for a debridement procedure. At the time of the second surgery, the infected rabbits were divided randomly into 3 subgroups for treating the infection. The osteomyelitic group was treated with 5 ml of sterile mPEG-PLGA containing teicoplanin (840 μg/mL) or no antibiotic, or treated with sterile PMMA cement beads containing teicoplanin. PMMA cement beads were prepared by mixing 40 g of cement (Zimmer Inc., Warsaw, Ind.) with 0.6 g of teicoplanin. The resulting mixture was formed into beads approximately 4 mm in diameter, weighing approximately 0.15 g. Two beads containing a total antibiotic dose of 4.5 mg were placed into each femur for the treatment.

After four weeks and eight weeks, bone specimens were sampled from affected region of the rabbits of the 3 subgroups. The bone specimens were fixed with 10% neutral buffered formalin. Histological specimens were decalcified, embedded in paraffin, and sections were stained with hematoxylin and eosin stain (H&E stain) and Gram's stain. The slides were evaluated by the histological grading scale, which contains three categories of investigation, including presence of bacteria, intraosseous inflammation and new bone formation. In each category the score was from 0 (healed) to 3 (worst) according to the severity of infection.

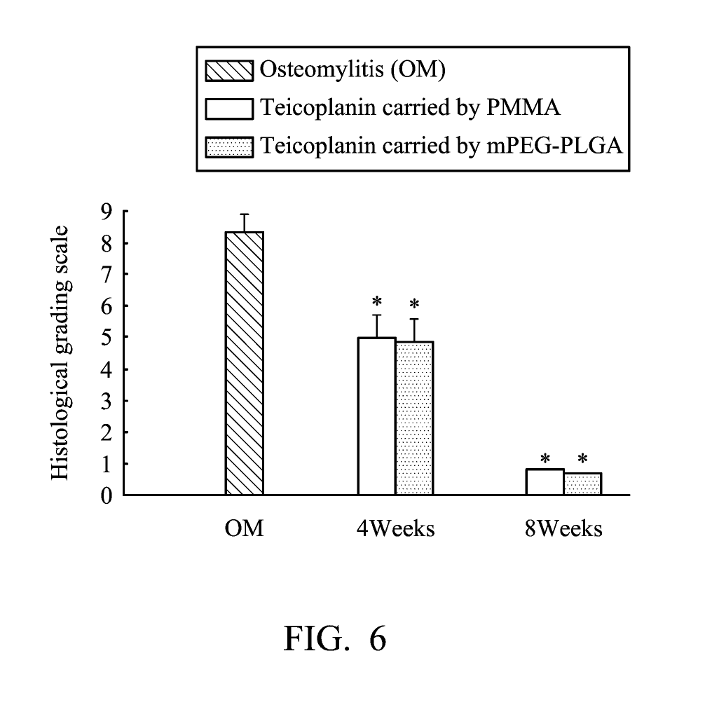

As shown in FIG. 6, the untreated subgroup exhibited a high histological grading scale. After a 4-week treatment with teicoplanin-impregnated PMMA or mPEG-PLGA, mixed inflammatory cell filtration was still observed in marrow. An almost thorough recovery from the bone infection was shown after an 8-week treatment.

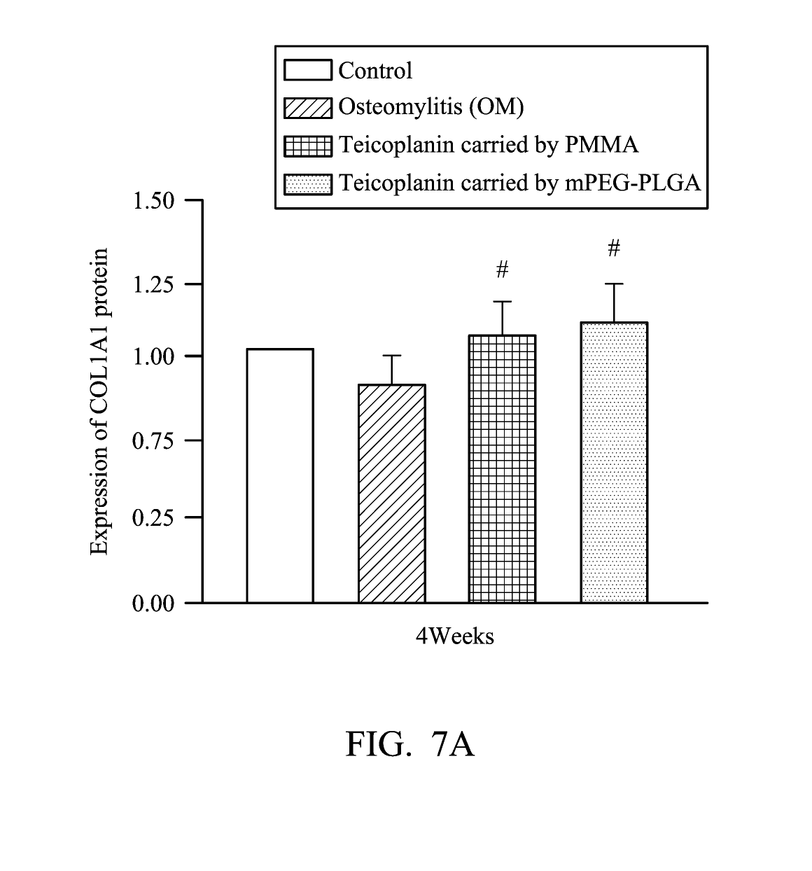

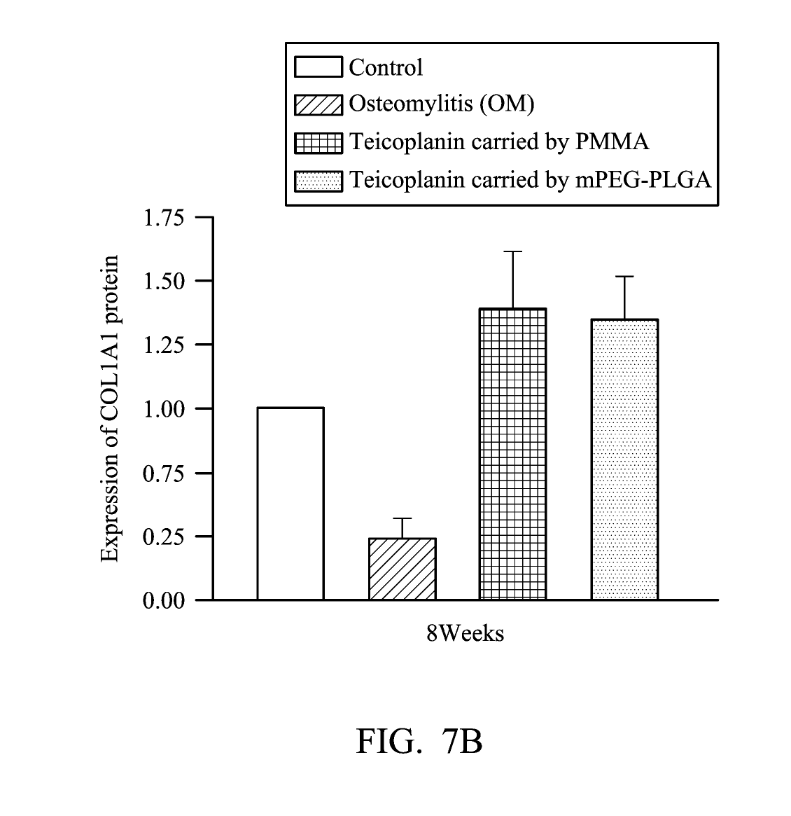

The expression of the COL1A1 gene was also evaluated in the staphylococcus aureus-infected rabbits and the infected rabbits received with teicoplanin treatment. The COL1A1 gene encodes the pro-alpha 1 chain of type 1 collagen, which is the main component for bone development and for bone reconstitution after healing from osteomyelitis. After 4 weeks, there were no statistic differences in the COL1A1 expression between untreated and teicoplanin-treated groups, as shown in FIG. 7A. However, both PMMA and mPEG-PLGA-treated groups had significantly elevated expression of the COL1A1 protein compared to the untreated group after 8 weeks, as shown in FIG. 7B.

Antibiotic-impregnated PMMA bone cements that provide high local concentration of antibiotic have been proved effective in treating osteomyelitis. However, the PMMA bone cements were not biodegradable and a second surgery is required to remove the cement beads to prevent them from becoming a nidus for infection. In addition to these disadvantages, the PMMA cement beads create some physical barriers that prevented new bone from growing into the defect. Compared to the bone cements, the composition of the disclosure can mitigate the aforementioned problems due to biodegradability. Compared to the other biodegradable polymers, the composition of the disclosure possesses several additional advantages, including easy preparation, high encapsulation efficiency of drugs or bioactive molecules, and freedom from harmful organic solvents in the formulation process. Further, the composition of the disclosure is released in a near-linear manner.

While the disclosure has been described by way of example and in terms of preferred embodiment, it is to be understood that the disclosure is not limited thereto. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A composition for the treatment of osteomyelitis, comprising:
100 parts by weight of water;
0.06-0.1 parts by weight of an antibiotic; and
5-40 parts by weight of a biodegradable thermosensitive polymer, wherein the biodegradable thermosensitive polymer has a structure as following: 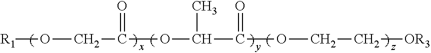
wherein, R₁ is hydrogen, or —C(═O)—R₂; R₂ is C₇₋₃₀ alkyl substituted or unsubstituted with functional groups; R₃ is hydrogen, or C₁₋₆ alkyl; and x, y or z individually are integers greater than 0.

2. The composition as claimed in claim 1, wherein the antibiotic comprises teicoplanin, vancomycin, telavancin, neomycin, or tobromycin.

3. The composition as claimed in claim 1, wherein the ration between x+y and z is from 1.8 to 2.8.

4. The composition as claimed in claim 1, wherein the composition exhibited a solution state below 10° C. and above 50° C., and the composition exhibited a sol-gel state between 10-50° C.

5. A method for preparing a composition for the treatment of osteomyelitis, comprising:
mixing the following components:
100 parts by weight of water;
0.06-0.1 parts by weight of an antibiotic; and
5-40 parts by weight of a biodegradable thermosensitive polymer, wherein the biodegradable thermosensitive polymer has a structure as following: 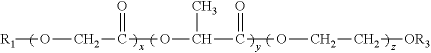
wherein, R₁ is hydrogen, or —C(═O)—R₂; R₂ is C₇₋₃₀ alkyl substituted or unsubstituted with functional groups; R₃ is hydrogen, or C₁₋₆ alkyl; and x, y or z individually are integers greater than 0.

6. A method for the treatment of osteomyelitis, comprising:
intramuscularly administering the composition of claim 1 to a subject suffering from osteomyelitis under conditions such that said osteomyelitis is reduced.
